(19) United States
(12) Patent Application Publication
(10) Pub. No.: US 2008/0038332 A1
(43) Pub. Date: Feb. 14, 2008

(54) Stable pharmaceutical formulation comprising atorvastatin calcium

(76) Inventors: Cai Gu Huang, Shang Gao (CN); Hui Min He Huang, Shang Gao (CN)

Correspondence Address: Hui Min He Huang, P.O. Box 193, Northborough, MA, 01532, US

(21) Appl. No.: 11/891,243

(22) Filed: Aug. 10, 2007

Related U.S. Application Data
(60) Provisional application No. 60/836,669, filed on Aug. 10, 2006.

(51) Int. Cl. A61K 9/48; A61K 31/07; A61K 31/355; A61K 31/40; A61K 33/00; A61K 33/06; A61K 9/14; A61K 9/20

(52) U.S. Cl. 424/451; 424/464; 424/489; 424/602; 424/610; 424/682; 514/428; 514/458; 514/725

(57) ABSTRACT

The invention relates to a stable pharmaceutical formulation comprising an intimate admixture or admixture of crystalline or amorphous atorvastatin calcium, and a stabilizing-effective amount of a water-insoluble alkaline excipient or a combination of one or more water-insoluble alkaline excipients thereof, a stabilizing-effective amount of an antioxidant or a combination of one or more antioxidants thereof, and at least one or more additional pharmaceutically acceptable inert excipients or carriers, and a method for the preparation of the said formulation by wet and dry granulation. The invention further relates to a stabilized intimate admixture of atorvastatin calcium, a water-insoluble alkaline excipient and an antioxidant and a method for the preparation of the said intimate admixture by co-precipitation and co-milling.

This application claims the benefit of priority to U.S. Provisional Patent Application Ser. No. 60/836,669, filed on Aug. 10, 2006, the entire disclosure of which is hereby incorporated by reference.

### FIELD OF THE INVENTION

The invention relates to a stable pharmaceutical formulation comprising crystalline or amorphous atorvastatin calcium, a stabilizing-effective amount of a water-insoluble alkaline excipient and an antioxidant, and at least one or more additional pharmaceutically acceptable inert excipients or carriers, and a method for the preparation of the same and a method for the therapeutic treatment using the same. The invention further relates to a method for the preparation of an intimate admixture of atorvastatin calcium, a water-insoluble alkaline excipient and an antioxidant.

### BACKGROUND OF THE INVENTION

Atorvastatin calcium has a chemical name of ((R—(R*, R*)-2-(4-fluorophenyl)-βδ-dihydroxy-5-(1-methylethyl)-3-phenyl-4[(phenylamino)-carbonyl]-1H-pyrrole-1-heptanoic acid, calcium salt (2:1), and its structure formula is shown below:

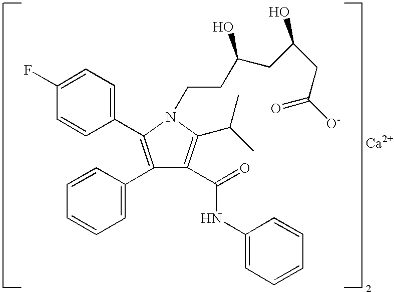

Atorvastatin hydroxyl acid form and its lactone form were first disclosed in U.S. Pat. No. 4,681,893, and its hemi calcium salt—atorvastatin calcium is disclosed in U.S. Pat. No. 5,273,995. Atorvastatin and pharmaceutically acceptable salts thereof is a well-known lipid-lowering agent, and they are inhibitors of 3-hydroxy-3-methylglutaryl-coenzyme A (HMG-CoA) reductase, which are especially useful in the treatment of hypercholesterolemia and hyperlipidemia.

Atorvastatin calcium has good stability and some formulation advantages over its other salt forms, the commercially available product, sold using trademark of Lipitor, contains crystalline atorvastatin calcium trihydrate (form I) as the active ingredient.

Atorvastatin calcium is a white to off-white powder that is very slightly soluble in distilled water, slightly soluble in ethanol, and freely soluble in methanol. It has been found amorphous atorvastatin calcium is more soluble in aqueous solutions than crystalline forms thereof. Therefore amorphous atorvastatin calcium or micronized material may have advantages in enhancing aqueous solubility, dissolution rate or even bioavailability.

U.S. Pat. Nos. 5,003,080; 5,097,045; 5,103,024; 5,124,482; 5,149,837; 5,155,251; 5,216,174; 5,245,047; 5,248,793; 5,280,132; 5,280,126; 5,342,952; 5,397,792; 5,007,080; 6,274,740; which are herein incorporated by reference, describe various processes and key intermediates for preparing atorvastatin calcium. All of these processes give mixture of crystalline and amorphous forms.

There have been found that atorvastatin calcium can exist in multiple crystal and amorphous forms.

A large number of patents applications or patents, EP 1659110, WO2006048888, WO2006046109, WO2006045018, WO2006039441, WO 200621959, WO2006011155, WO2005100313, WO2005092852, US2005165242, WO2005005384, US2004242670, WO2004089895, WO2004085391, WO2003099785, US2006128971, US2004186162, US2005131055, US2005119493, U.S. Pat. No. 6,750,353, U.S. Pat. No. 6,613,916 and U.S. Pat. No. 6,891047, WO 97/03 960 and WO 00/71116, which are herein incorporated by reference describe various processes for the preparation of amorphous atorvastatin calcium. The amorphous atorvastatin calcium was generally prepared by dissolving a crude or crystalline atorvastatin calcium in various polar solvents and then precipitating out amorphous atorvastatin calcium by adding anti solvents (non polar solvents) or by removing the polar solvents with spray-drying, thin-film drying, vacuum drying or freeze-drying techniques.

More than 40 crystalline polymorph forms of atorvastatin calcium have been described so far in publications. Thus WO 97/03958 as well as WO 97/03959 filed at the same time describe the crystalline forms III as well as I, II and IV (also see U.S. Pat. No. 5,969,156) of atorvastatin calcium.

WO 01/36384 discloses the polymorph form V of atorvastatin calcium which is also obtained from a methanol-water mixture.

WO 02/41834 describes the polymorph form VII of atorvastatin calcium which can be obtained by stirring a suspension of the polymorph form V or the polymorph form I of atorvastatin calcium in absolute ethanol at room temperature.

WO 02/43732 discloses the polymorph forms VI, VIII, IX, X, XI and XII of the atorvastatin calcium. WO 02/51804 describes the polymorph forms X, A, B1, B2, C, D and E of the atorvastatin calcium.

WO 03/04470 describes the polymorph forms V, VI, VII, VIII, IX, X, XI, XII, XIII, XIV, XV, XVI, XVII, XVIII and XIX of atorvastatin calcium. WO06011041 discloses the preparation of crystalline atorvastatin calcium Form XX, Form XXI, Form XXIII, Form XXIV, Form XXIV, Form XXV, Form XXVI, Form XXVII, Form XXVIII, Form XXIX, and Form XXX.

Different crystalline polymorphs or amorphous form of the same compound have different physical properties and chemical stability. One of the most important physical properties of pharmaceutical polymorphs is their solubility or dissolution rate in aqueous solution. Other important properties are their chemical stability, which are very relevant in the case of atorvastatin calcium since it is chemically unstable.

Atorvastatin calcium is very susceptible to decomposition when it is exposed to oxygen, light, temperature, humidity, carbon dioxide and acidic pharmaceutical excipients. For instance, atorvastatin calcium decomposes very rapidly to form atorvastatin lactone under acidic environment. In addition, atorvastatin calcium, particularly amorphous atorvastatin calcium, can be readily oxidized under an atmosphere containing oxygen or in the presence of oxidizing agents to form several undesirable degradation products.

U.S. Pat. Nos. 5,686,104 and 6,126,971 disclose stable solid formulations comprising atorvastatin calcium and an alkaline earth metal salt as a stabilizer. These documents teach that pharmaceutical formulation comprising atorvastatin calcium will exhibit improved stability, for example, to inhibit or slow down atorvastatin lactone formation, when the formulation comprises an alkaline earth metal salt such as calcium carbonate, calcium hydroxide, magnesium carbonate, magnesium silicate, magnesium aluminate and aluminum magnesium hydroxide. However, these patents do not specify whether the atorvastatin calcium being in the examples used is the amorphous form or crystalline forms. It appears that the atorvastatin calcium used in the examples in these two patents is crystalline form. It has been found that amorphous atorvastatin calcium is much less stable than crystalline forms, particularly towards to oxidative degradation, and therefore the formulations within the scope of U.S. Pat. Nos. 5,686,104 and 6,126,971 do not enable adequate stability of amorphous atorvastatin calcium. Additionally, these patents make readers to believe that only an alkaline earth metal salt such as calcium carbonate is able to stabilize atorvastatin calcium.

U.S. Pat. No. 6,680,341 teaches a method to stabilize statin compounds such as pravastatin sodium by incorporating a small amount of water soluble buffer agents into the active ingredient, to inhibit or slow down the formation of statin lactone, a common degradant formed from the ring closure of hydroxyl acids. Most examples in this disclosure deal with pravastatin sodium, and only example 6 deals with atorvastatin calcium. It is more difficult to provide stable formulation for atorvastatin calcium than for pravastatin sodium. Unlike provastatin sodium, atorvastatin calcium, particularly amorphous atorvastatin calcium, is not only very unstable toward acid excipients, but also very susceptible to oxidative degradation. The statement made in example 6 of this disclosure is not consistent with observations made by other publications, most likely due to that oxidative degradants were not measured in U.S. Pat. No. 6,680,341. As disclosed in WO2006/008091, oxidative degradants are eluted earlier than atorvastatin in reverse phase HPLC and have much less responses at some UV wavelengths. Therefore, this patent fails to teach stable pharmaceutical formulations comprising atorvastatin calcium, particularly amorphous atorvastatin calcium and methods to make such stable formulations.

Publications PCT WO03/097039 and EP 1336405 describe stable formulations comprising amorphous atorvastatin calcium and a water-soluble alkaline metal additive or stabilizer such as sodium or potassium compounds. The additives or stabilizers disclosed in these publications are limited to sodium carbonate, sodium hydroxide, sodium hydrogen phosphate, sodium dehydrogen phosphate, sodium phosphate, potassium hydroxide, potassium carbonate and potassium tribasic phosphate. In examples of EP 1336405, formulations comprising sodium carbonate and disodium hydrogen orthophosphate as additives were described. In the stability evaluation, only assay of active ingredient was measured, and total impurities, including lactone and oxidative degradants, were not measured. Therefore, this publication fails to disclose the effectiveness of these additives in preventing or inhibiting the formation of atorvastatin calcium degradants.

In PCT WO03/097039, a stability study was conducted for compositions containing amorphous atorvastatin calcium and a water-soluble stabilizer such as sodium carbonate monohydrate, sodium phosphate dibasic anhydrous or tribasic sodium phosphate anhydrous. It concluded that tribasic sodium phosphate is the only effective stabilizer. However, the compositions used in the stability study contain too much stabilizer, and the weight ratio of active ingredient to stabilizer is 5.4 to 125.6. According to this weight ratio, 40 or 80 mg strength atorvastatin calcium tablets and capsules would weigh over 1 or 2 grams for each dosage unit, too large for conventional solid dosage units. Additionally, the condition used to evaluate the stability of atorvastatin calcium is 80° C., and the stability data obtained under such high temperature may be not relevant to those obtained under normal storage conditions. It has been found that amorphous atorvastatin calcium is very susceptible toward oxidation, and a water-soluble alkaline stabilizer alone cannot adequately stabilize it in solid dosage forms, particularly when a strong water soluble base such as sodium phosphate is used and the pH of the formulation approaches to 11, creating a very strong basic environment which would facilitate an oxidative degradation of atorvastatin calcium.

WO 04/032920 and WO 05/011638 describes stabilization of amorphous atorvastatin calcium formulation by packaging the dosage forms under an inert gas (e.g., nitrogen or argon) atmosphere or under a gaseous mixture with a partial pressure of oxygen less than 2 kPa or using oxygen absorbents to reduce the oxygen level in the packaging containers. The drawbacks of these stabilizing methods are that they need special blisters to package the products, and that oxygen molecule will continuously penetrate into the containers when the pressure in inside container is lower than atmosphere, and that patients may accidentally take oxygen absorbents which are usually very toxic.

WO 01/76566 discloses stabilization of atorvastatin calcium by using polymers comprising at least one amino group or at least one amido group. The composition can only provide moderate stability effect against lactone formation, and such polymers cannot provide any stability effect on oxidative degradation of atorvastatin calcium, particularly amorphous atorvastatin calcium. Therefore, this publication also fails to provide formulations and methods for stabilizing atorvastatin calcium, particularly amorphous atorvastatin calcium.

Finally, WO06/054308 discloses a pharmaceutical formulation comprising atorvastatin calcium without any stabilizers. It describes a drug-excipient compatibility study using atorvastatin calcium (both crystalline and amorphous forms) and starch 1500, lactose monohydrate, mannitol, CaCO₃, Avicel 101, croscamellose sodium, crospovidone, sodium starch glycolate, carmellose calcium and stearic acid. The essence of this disclosure is that acid excipient such as stearic acid, and ionic or salt excipients such as croscamellose sodium, carmellose calcium and sodium starch glycolate are incompatibility with atorvastatin calcium, which is already known in the prior art. In addition, the weight ratio of active ingredient to excipient is 1 to 5 is not applicable to the practical formulations. Croscamellose sodium, carmellose calcium and sodium starch glycolate are disintegrants and their suitable concentrations employed in formulations are generally between 2% and 8%. At such level, these disintegrants are essentially compatible with atorvastatin calcium, particularly in the presence of excipient such as calcium carbonate. For example, Lipitor contains croscamellose sodium and it is still very stable drug product. As described in compatibility studies of this disclosure, oxidative degradants were not measured or not appropriately measured in WO06/054308. As disclosed in WO2006/008091, oxidative degradants are eluted earlier than atorvastatin in reverse phase HPLC and have much less response at some UV wavelengths. Therefore, this publication does not teach formulations and methods for stabilizing atorvastatin calcium, particularly amorphous atorvastatin calcium.

All of these approaches in the prior art to prevent the chemical degradation of atorvastatin calcium, particularly amorphous atorvastatin calcium, in solid dosage forms have a number of clear drawbacks and disadvantages. Therefore, there is a constant need for a stable pharmaceutical formulation comprising atorvastatin calcium, particularly amorphous atorvastatin calcium.

### SUMMARY OF THE INVENTION

The applicant has discovered that a new class of water-insoluble alkaline excipients and a new class of antioxidants are able to effectively prevent or significantly inhibit lactone formation and oxidative degradations of atorvastatin calcium, particularly when they are intimately admixed with amorphous or crystalline atorvastatin calcium in pharmaceutical formulations.

Therefore, according to one aspect of the present invention, there is a stable pharmaceutical formulation comprising an intimate admixture or admixture of amorphous or crystalline atorvastatin calcium or its pharmaceutically acceptable solvates or hydrates, as active ingredient, a stabilizing-effective amount of an water-insoluble alkaline excipient or optionally a combination of two or more said excipients thereof, and a stabilizing-effective amount of antioxidant or optionally a combination of two or more said antioxidants thereof, and at least one or more additional pharmaceutically acceptable inert excipients or carriers.

The said water-insoluble alkaline excipients are selected from a group consisting of zinc compounds, including zinc carbonate, zinc dibasic phosphate, zinc tribasic phosphate (zinc phosphate), cobalt compounds, including cobalt carbonate, cobalt tribasic phosphate (cobalt phosphate), glycerophosphate compounds, including calcium glycerophosphate, magnesium glycerophosphate, sodium glycerophosphate and potassium glycerophosphate, magnesium compounds, including magnesium dibasic phosphate, magnesium tribasic phosphate (magnesium phosphate) and magnesium carbonate hydroxide or calcium tribasic phosphate (calcium phosphate).

The said antioxidants are selected from a group consisting of ascorbate compounds, including potassium ascorbate, calcium ascorbate, ascorbyl palmitate and ascorbyl stearate, vitamin derivatives, including vitamin A, vitamin A₂, natural and synthetic tocopherols (including mixed tocopherols concentrate, alpha-tocopherol, beta-tocopherol, synthetic gamma-tocopherol, synthetic delta-tocopherol), vitamin E, propyl gallate, tertiary butyl hydroquinone (TBHQ), 2,4,5-trihydroxybutyrophenone, dilauyl thiodipropionate, magnesium sulfite, calcium sulfite. The said antioxidants further include antioxidant synergists, including sodium citrate, potassium citrate, magnesium citrate, calcium citrate or diaminoethanetraacetic acid disodium salt (EDTA).

According to another aspect of the present invention, there is a method for preparation of a stable pharmaceutical formulation comprising amorphous or crystalline atorvastatin calcium or its pharmaceutically acceptable solvates or hydrates, comprising steps of admixing or intimately admixing a stabilizing-effective amount of a water-insoluble alkaline excipient or optionally a combination or two or more said excipients thereof, and a stabilizing-effective amount of an antioxidant or optionally a combination of two or more said antioxidants thereof, and amorphous or crystalline atorvastatin calcium, and further mixing the said admixture or intimate admixture with at least one or more additional pharmaceutically acceptable inert excipients or carriers by wet granulation or dry granulation such as compacting to obtain the stable pharmaceutical preparation.

According to another aspect of the present invention, there is a stabilized intimate admixture comprising of atorvastatin calcium, a stabilizing-effective amount of one or more water-insoluble alkaline excipients and a stabilizing-effective amount of one or more antioxidants.

According to still another aspect of the present invention, there is a method for preparation of an intimate admixture of atorvastatin calcium, a water-insoluble alkaline excipient and an antioxidant by co-precipitation and co-milling.

According to a further aspect of the present invention, there is a method for the treatment of hypercholesterolemia and hyperlipidemia, comprising the step of orally administering to a patient in need of such treatment a therapeutically effective unit dosage of a stabilized pharmaceutical formulation comprising amorphous or crystalline atorvastatin calcium or pharmaceutically acceptable solvates or hydrates, a stabilizing-effective amount of a water-insoluble alkaline excipient or optionally a combination of two or more said excipients thereof, and a stabilizing-effective amount of an antioxidant or a combination or two or more said antioxidants thereof, and plus at least one or more additional pharmaceutically acceptable inert excipients or carriers.

### DETAILED DESCRIPTION OF THE INVENTION

As described above, atorvastatin calcium, particularly amorphous atorvastatin calcium is very unstable, and is susceptible to acid-catalyzed hydroxyl acid ring closure degradation, and light-induced or oxygen-induced or other oxidizing agents induced oxidative degradation, leading to form lactone and numerous other degradants. In a research to prevent or inhibit such degradations, the applicant has surprisingly found that a new class of poorly water soluble or water-insoluble alkaline excipients, when they are combined with a new class of antioxidants, especially if they are present as an intimate admixture with atorvastatin calcium as obtained by co-precipitate, co-milling, roller compacting and wet granulation, is able to effectively prevent or significantly inhibit atorvastatin calcium degradations. As a result, a new stabilized atorvastatin calcium pharmaceutical formulation can be made with the said alkaline excipients thereof, the said antioxidant thereof and with at least one or more pharmaceutically acceptable inert excipients or carriers.

The acid-catalyzed hydroxyl acid ring closure degradation of atorvastatin calcium is governed by pH of the local environment in the dosage forms. Water soluble alkaline stabilizers or additives disclosed in prior art can dissolve in a local and micro environment of dosage forms to form a liquid to remove the undesirable acids, leading to the stabilization of atorvastatin calcium. However, it is believed that water-insoluble alkaline excipients is not able to form such liquid in dosage forms and thus it cannot effectively remove undesirable acids and stabilize atorvastatin calcium. Therefore, the stabilizating effect of the water-insoluble alkaline excipients on pharmaceutical formulation comprising atorvastatin calcium is not predictable.

The advantages and benefits of the present invention is that the poorly water soluble or water-insoluble alkaline excipients will not rapidly dissolve in the local acidic stomach environment, and thus does not create a sudden disturbance in the patient's stomach, while those metal or earth metal alkaline stabilizers or buffer agents disclosed in prior art are either highly water soluble or water soluble, and they will rapidly dissolve in the acidic stomach and disturb the patient' stomach. Additionally, the present invention provides new approaches to stabilize atorvastatin calcium in pharmaceutical formulations by making an intimate admixture of the water-insoluble alkaline excipients and the antioxidants. The prior art does not teach or suggest the use of intimate admixture of water-insoluble alkaline excipients (or any other alkaline additives) and antioxidants to stabilize amorphous or crystalline atorvastatin calcium in pharmaceutical formulation. Furthermore, water-insoluble alkaline excipients and antioxidants disclosed in present invention are previously unrecognized in the prior art, and therefore the present invention provides new ways to formulate stable atorvastatin calcium dosage forms.

Accordingly, the present invention provides a stable pharmaceutical formulation comprising amorphous or crystalline atorvastatin calcium or pharmaceutically acceptable solvates or hydrates thereof, as the active ingredient, and a stabilizing-effective amount of a water-insoluble alkaline excipient or optionally a combination of two or more said excipients thereof, a stabilizing-effective amount of an antioxidant or optionally a combination or two or more said antioxidants thereof, and at least one or more additional pharmaceutically acceptable inert excipients or carriers.

Preferably, the present invention provides a stable pharmaceutical formulation comprising an admixture of amorphous or crystalline atorvastatin calcium or pharmaceutically acceptable solvates or hydrates thereof, as the active ingredient, and a stabilizing-effective amount of a water-insoluble alkaline excipient or optionally a combination of two or more said excipients thereof, and a stabilizing-effective amount of an antioxidant or optionally a combination or two or more said antioxidants thereof, and at least one or more additional pharmaceutically acceptable inert excipients or carriers.

More preferably, the present invention provides a stable pharmaceutical formulation comprising an intimate admixture of amorphous or crystalline atorvastatin calcium or pharmaceutically acceptable solvates or hydrates thereof, as the active ingredient, and a stabilizing-effective amount of a water-insoluble alkaline excipient or optionally a combination of two or more said excipients thereof, and a stabilizing-effective amount of an antioxidant or optionally a combination or two or more said antioxidants thereof, and at least one or more additional pharmaceutically acceptable inert excipients or carriers.

The term “stabilizing-effective amount,” used in reference to the amount of a water-insoluble alkaline excipient or an antioxidant in the stabilized pharmaceutical formulation comprising amorphous or crystalline atorvastatin calcium, means an amount such that no more than about 3.0%, preferably no more than 2.0%, and most preferably no more than 1.5% by weight of atorvastatin calcium in the stabilized atorvastatin calcium pharmaceutical formulations is degraded upon exposure to 40° C./75% relative humidity (RH) for three (3) months.

The term “stabilizing-effective amount,” used in reference to the amount of a water-insoluble alkaline excipient or an antioxidant in the stabilized pharmaceutical formulation comprising amorphous or crystalline atorvastatin calcium, means an amount such that no more than about 2.0%, preferably no more than 1.5%, and most preferably no more than 1.0% by weight of total degradation products from atorvsattin calcium in the stabilized pharmaceutical formulations is formed upon exposure to 40° C./75% relative humidity for three (3) months.

The term “stabilizing-effective amount,” used in reference to the amount of a water-insoluble alkaline excipient or an antioxidant in the stabilized intimate admixtures comprising amorphous or crystalline atorvastatin calcium, means an amount such that no more than about 1.5%, preferably no more than 1.0%, and most preferably no more than 0.7% by weight of total degradation products from atorvastatin calcium in the stabilized intimate admixtures is formed upon exposure to 40° C./75% relative humidity for three (3) months.

The term “water-insoluble alkaline excipients”,” used in reference to excipients with a pKₐ of the conjugated acid of at least 2.5 and a Kₛₚ of about 1×10⁻¹ to about 1×10⁻¹⁵, usually about 1×10⁻² to about 1×10⁻¹¹. The said water-insoluble alkaline excipients of the present invention also includes slightly water soluble alkaline excipients, sparingly water soluble alkaline excipients, very slightly water soluble alkaline excipients and poorly water soluble alkaline excipients,

The said water-insoluble alkaline excipients of the present invention are selected from a group consisting of zinc compounds, including zinc carbonate, zinc carbonate hydroxide, zinc dibasic phosphate, zinc tribasic phosphate (zinc phosphate), or cobalt compounds, including cobalt carbonate, cobalt carbonate hydroxide, cobalt tribasic phosphate (cobalt phosphate), glycerophosphate compounds, including calcium glycerophosphate, magnesium glycerophosphate, sodium glycerophosphate (disodium glycerophosphate) and potassium glycerophosphate (dipotassium glycerophosphate), magnesium compounds, including as magnesium dibasic phosphate, magnesium tribasic phosphate (magnesium phosphate) and magnesium carbonate hydroxide, or citrate compounds, including calcium citrate, magnesium citrate, or calcium tribasic phosphate (calcium phosphate).

Preferably, the suitable water-insoluble alkaline excipients of the present invention are selected from a group consisting of zinc carbonate, zinc carbonate hydroxide, zinc dibasic phosphate, zinc tribasic phosphate (zinc phosphate), cobalt carbonate, cobalt carbonate hydroxide, cobalt tribasic phosphate (cobalt phosphate), calcium glycerophosphate, magnesium glycerophosphate, sodium glycerophosphate (disodium glycerophosphate) and potassium glycerophosphate (dipotassium glycerophosphate), magnesium dibasic phosphate, magnesium tribasic phosphate (magnesium phosphate) and magnesium carbonate hydroxide, calcium citrate, magnesium citrate, or calcium tribasic phosphate (calcium phosphate).

More preferably, the suitable water-insoluble alkaline excipients of the present invention are selected from a group consisting of zinc carbonate, zinc dibasic phosphate, zinc tribasic phosphate (zinc phosphate), cobalt carbonate, cobalt tribasic phosphate (cobalt phosphate), calcium glycerophosphate, magnesium glycerophosphate, magnesium dibasic phosphate, magnesium tribasic phosphate (magnesium phosphate) and magnesium carbonate hydroxide, calcium citrate, magnesium citrate, or calcium tribasic phosphate (calcium phosphate).

The said antioxidants of the present invention are selected from a group consisting of ascorbate compounds, including potassium ascorbate, calcium ascorbate and magnesium ascorbate, vitamin derivatives, including vitamin A, vitamin A₂, natural and synthetic tocopherols (mixed tocopherols concentrate, alpha-tocopherol, beta-tocopherol, synthetic gamma-tocopherol, synthetic delta-tocopherol), vitamin E, ascorbyl palmitate, ascorbyl stearate, propyl gallate, tertiary butyl hydroquinone (TBHQ), dilauyl thiodipropionate, magnesium sulfite and calcium sulfite. The said antioxidants further include antioxidant synergists which are selected from a group consisting of sodium citrate, potassium citrate, magnesium citrate, calcium citrate or diaminoethanetraacetic acid disodium salt (EDTA). Preferably, the antioxidant is a food grade antioxidant, however any antioxidant, which is generally recognized as pharmaceutically acceptable, may be used.

The amorphous atorvastatin calcium of the present invention can be pure amorphous or a mixture of amorphous and crystalline materials, preferably pure amorphous form. The said amorphous atorvastatin calcium also includes amorphous anhydrous, amorphous solvates or amorphous hydrates, which can be obtained according to the references as described above.

The amorphous atorvastatin calcium can be an amorphous solid solution with any suitable carriers such as PEGs, an amorphous solid dispersion with any suitable carriers or polymers, or an amorphous co-precipitate with any suitable excipients.

The term “amorphous” means a solid without long-range crystalline order. Amorphous form of atorvastatin calcium in accordance with the present invention preferably contains less than about 20% crystalline forms of atorvastatin calcium, preferably less than 10% crystalline form of atorvastatin calcium, and more preferably less than 2% crystalline form of atorvastatin calcium, and most preferably is essentially free of crystalline forms of atorvastatin calcium. “Essentially free of crystalline forms of atorvastatin calcium” means that no crystalline forms of atorvastatin calcium can be detected within the limits of a powder X-ray diffractometer.

The said crystalline atorvastatin calcium of the present invention can be any crystalline polymorph forms, including, but not limited to, form I, II, III, IV, V, VI, VII, VIII, IX, X, XI, XII, XIII, XIV, XV, XVI, XVII, XVIII and XIX or any other polymorphs of crystalline atorvastatin calcium. Crystalline atorvastatin calcium can be anhydrous, hydrates or pharmaceutically acceptable solvates, which can be obtained according the references as described above.

Accordingly, in a preferred embodiment, the present invention provides a stable pharmaceutical formulation comprising an admixture or an intimately admixture of amorphous atorvastatin calcium or pharmaceutically acceptable solvates or hydrates thereof, as the active ingredient, a stabilizing-effective amount of a water-insoluble alkaline excipient or a combination of two or more said excipients thereof, and a stabilizing-effective amount of an antioxidant or a combination or two or more said antioxidants thereof, and at least one or more additional pharmaceutically acceptable inert excipients or carriers.

Accordingly, in another preferred embodiment, the present invention provides a stable pharmaceutical formulation comprising an admixture or an intimately admixture of crystalline atorvastatin calcium or pharmaceutically acceptable solvates or hydrates thereof, as the active ingredient, and a stabilizing-effective amount of a water-insoluble alkaline excipient or a combination of two or more said excipients thereof, and a stabilizing-effective amount of an antioxidant or a combination or two or more said antioxidants thereof, and at least one or more additional pharmaceutically acceptable inert excipients or carriers.

A combination of two or more water-insoluble alkaline excipients can be selected from a group consisting of any above said water-insoluble alkaline excipients. Preferably, the combination of two or more water-insoluble alkaline excipients is able to effectively prevent or inhibit the hydroxyl acid ring closure degradation (to form lactone) to significantly inhibit or slow down the oxidative decomposition of atorvastatin calcium as well, when they are admixed or intimately admixed with antioxidants or antioxidants synergists.

A combination of two or more antioxidants can be selected from a group consisting of any above said antioxidants or antioxidant synergists. Preferably, the combination of an antioxidant and antioxidant synergist is able to effectively prevent or inhibit the oxidative degradation and the hydroxyl acid ring closure (to form lactone) reaction, when they are admixed or intimately admixed with suitable water-insoluble alkaline excipients, thus leading to significantly overall slow down decomposition rate of atorvastatin calcium.

The said one or more additional pharmaceutically acceptable inert excipients or carriers of the present invention can be any inert excipients or carriers, for example, a binder, a filler or diluent, a disintegrant, a surfactant, flavoring agents, a lubricant or a glidant, and can be either solid or liquid, preferably a solid. The said inert excipient or carrier means that it is chemically and physically compatible with atorvastatin calcium when they are formulated in a pharmaceutical preparation. In such preparation, an inert excipient or carrier does not considerably cause or accelerate the physical and chemical changes of the active ingredient.

In a preferred embodiment of the present invention, the water-insoluble alkaline excipient in the pharmaceutical formulations is present in an amount that can effectively prevent or significantly slow down the decomposition of the active ingredient or considerably inhibit the formation of degradation products by stabilizing the active ingredient in the pharmaceutical preparations. The water-insoluble alkaline excipient may be present at a concentration of between about 0.5% and about 50% by weight of the formulation, preferably at a concentration of between about 1.0% and about 30% by weight of the formulation, more preferably at a concentration of between about 3.0% and about 20% by weight of the formulation.

In a preferred embodiment of the present invention, the antioxidant or antioxidant synergist in the pharmaceutical formulations is present in an amount that can effectively prevent or significantly slow down the decomposition of the active ingredient or considerably inhibit the formation of oxidative or other degradation products by stabilizing the active ingredient in the pharmaceutical preparations. The antioxidant or antioxidant synergist may be present at a concentration of between about 0.01% and about 10% by weight of atorvastatin calcium, preferably at a concentration of between about 0.1% and about 5% by weight of atorvastatin calcium, more preferably at a concentration of between about 1.0% and about 3% by weight of atorvastatin calcium.

In another preferred embodiment of the present invention, when a combination of two or more water-insoluble alkaline excipients is included in the pharmaceutical formulation, they may be present in an amount that can effectively prevent or significantly slow down the decomposition of the active ingredient or considerably inhibit the formation of degradation products by stabilizing atorvastatin calcium in the pharmaceutical preparations. The total concentration of two or more water-insoluble alkaline excipients thereof may be present at about 0.5% to about 50% by weight of the formulation, preferably at about 1.0% to about 30% by weight of the formulation, more preferably at about 2.0% to about 25% by weight of the formulation. The weight ratio of two water-insoluble alkaline excipients may be present in any ratio, preferably at about 0.1:10 to about 10:0.1, more preferably about 0.5:2 to about 2:0.5, most preferably about 0.8:1.2 to about 1.2:0.8.

Alternatively, the concentration of the water-insoluble alkaline excipients in the formulation may be dependent on the concentration of the active ingredient in the formulation. When the concentration of the active ingredient changes, the concentration of the water-insoluble alkaline excipients should also be adjusted accordingly to maintain an appropriate ratio of the active ingredient to the water-insoluble alkaline excipients so that the water-insoluble alkaline excipients can effectively stabilize the active ingredient.

More specifically, the amount of the water-insoluble alkaline excipients which should be used to achieve the results desired, for stabilization of amorphous or crystalline atorvastatin calcium in pharmaceutical formulations, e.g., tablets and capsules is about 1%-500% by weight ratio of water-insoluble alkaline excipients/active ingredient, preferably about 20%-200% weight ratio of/active ingredient and most preferably 50%-150% by weight ratio of water-insoluble alkaline excipients/active ingredient.

The amount percentage of water-insoluble alkaline excipients is also dependent on their nature and molecular weights, as well as the label strength of atorvastatin calcium in pharmaceutical formulations, which can be readily determined by one skilled in art.

The term “pharmaceutical formulation” as used herein is intended to encompass a product including the active ingredient(s), any additional pharmaceutically acceptable excipients or carriers that make up the carrier, as well as any product which results, directly or indirectly, from combination, or aggregation of any two or more of the ingredients, or from dissociation of one or more of the ingredients, or from other types of reactions or interactions of one or more of the ingredients. Accordingly, the pharmaceutical formulations of the present invention encompass any composition made by admixing or intimately admixing the active ingredient, water-insoluble alkaline excipients, antioxidants or its synergists, optionally additional active ingredient, and at least one ore more pharmaceutically acceptable excipients or carriers.

In another referred embodiment of the present invention, the active ingredient, atorvastatin calcium, in pharmaceutical formulations is present in an amount that is therapeutically effective for the treatment of hypercholesterolemia and hyperlipidemia. The active ingredient, atorvastatin calcium may be present at a concentration of between about 1.0% and about 60% by weight of the formulation, preferably at a concentration of between about 5.0% and about 40% by weight of the formulation, more preferably at a concentration of between about 10% and about 30% by weight of the formulation, and most preferably, the pharmaceutical formulation may contain one or more of a 10 mg atorvastatin dosage unit, a 20 mg atorvastatin dosage unit, a 40 mg dosage unit, an 80 mg atorvastatin dosage unit.

The total amount of inactive ingredient in the pharmaceutical formulation, including the amount of water-insoluble alkaline excipients, antioxidants and pharmaceutically acceptable inert excipients or carriers, is preferably more than 50% of the weight of atorvastatin calcium in the composition and less than 2,000% of the weight of atorvastatin calcium.

According to another aspect of the present invention, there is a method for preparation of a stable pharmaceutical formulation comprising amorphous or crystalline atorvastatin calcium or its pharmaceutically acceptable solvates or hydrates, comprising steps of admixing, preferably intimately admixing a stabilizing-effective amount of a water-insoluble alkaline excipient or a combination or two or more such excipients, and a stabilizing-effective amount of an antioxidant or a combination of two or more such antioxidants, and amorphous or crystalline atorvastatin calcium, and followed by wet granulation or dry (roller compacting) granulation to admix the said intimate admixture with additional pharmaceutically inert excipients or carriers. The granules can then be filled into capsules or compressed into tablets or used in making other pharmaceutical dosage forms. The details of wet granulation and roller contacting dry granulation for making tablets are described in following sections and in the examples of the present invention.

According to another aspect of the present invention, there is a stabilized intimate admixture comprising of atorvastatin calcium, a stabilizing-effective amount of one or more water-insoluble alkaline excipients and a stabilizing-effective amount of one or more antioxidants.

According to still another aspect of the present invention, there is a method for preparation of an intimate admixture of atorvastatin calcium, a water-insoluble alkaline excipient and an antioxidant by co-precipitation, co-milling, compacting or wet granulation.

In one embodiment, an intimate admixture of atorvastatin calcium, a water-insoluble alkaline excipient and an antioxidant is made by co-precipitating method, comprising steps of adding a water-insoluble alkaline excipient, an antioxidant and atorvastatin calcium in a suitable organic solvent and then partially or completely evaporate the solvent to obtain an intimate admixture. Preferably, this embodiment comprises the steps of: 1) dissolving atorvastatin calcium, a water-insoluble alkaline excipient and an antioxidant in a suitable organic solvent; 2) evaporating the organic solvent to form a dry co-precipitate; 3) further drying the co-precipitate under vacuum oven to remove solvent residue to provide an co-precipitate of atorvastatin calcium, a water-insoluble alkaline excipient and an antioxidant. The co-precipitate comprises atorvastatin calcium, a water-insoluble alkaline excipient and an antioxidant in intimate admixture.

The preferred solvent in the disclosed methods is an alcohol or tetrahydrofuran (THF). More preferably, the solvent is a lower straight or branched-chain alkanol such as methanol, ethanol, propanol, isopropanol, etc.

In another embodiment, an intimate admixture of atorvastatin calcium, a water-insoluble alkaline excipient and an antioxidant is made by co-milling atorvastatin calcium, a water-insoluble alkaline excipient and an antioxidant. Co-milling may be done by grinding the atorvastatin calcium, a water-insoluble alkaline excipient and an antioxidant using conventional methods such as with a mortar and pestle or by co-micronizing the atorvastatin calcium, a water-insoluble alkaline excipient and an antioxidant.

The intimate admixture of the atorvastatin calcium, a water-insoluble alkaline excipient and an antioxidant, obtained by co-precipitating or co-milling, may then be formulated into suitable pharmaceutical formulations with conventional inert excipients or carriers as described above.

The pharmaceutical formulations of the present invention typically contain, in addition to the admixture or intimate admixture of atorvastatin calcium, a water-insoluble alkaline excipient and an antioxidant, at least one or more pharmaceutically acceptable inert excipients or carriers, such as binders, fillers, disintegrants, carriers, lubricants, glidants, flavorants, surfactants, colorants, buffers, thickening agents. Some excipients can serve multiple functions, for example as both binder and disintegrant.

The pharmaceutical formulation of the present invention is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Pharmaceutical formulations of the present invention may optionally include any inert ingredients for improving the dissolution rate, physical properties, visual appearance or odor of the pharmaceutical. The excipients are selected based on the desired physical aspects of the final form: e.g., obtaining a tablet with desired hardness and friability, being rapidly dispersible and easily swallowed, etc. The desired release rate of the active substance from the composition after its ingestion also plays a role in the choice of excipients. Preferred release rate is the rate comparable with commercially available Lipitor tablets.

Pharmaceutical formulations of the present invention include dosage forms such as tablets, granulates, hard or soft capsules, powders, solutions, emulsions, suspensions, or the like. Tablets are particularly preferred dosage forms of the pharmaceutical formulations in accordance with the present invention. Among the methods for forming preferred tablet dosage forms are included, e.g., wet granulation, dry granulation, e.g., compaction and slugging, and direct compression.

According to a preferred aspect of the present invention, the water content of the excipients is very low. More specifically, the water content in the diluents is very low in order to minimize the water content of the pharmaceutical composition. Lactose is used in its anhydrous form. Furthermore, all excipients may be used in a dry form. Preferably all excipients used for the preparation of the pharmaceutical formulation should have water content below about 3%, preferably below about 1%, more preferred below about 0.5% (weight/weight).

Typically atorvastatin calcium to be mixed is in the form of particles. The storage stability of the pharmaceutical formulation of the present invention is enhanced, in general, by using larger particle sizes. Preferably the average particle size of free atorvastatin calcium is at least 1 microns, more preferably at least 5 microns, and most preferably in a range from 5 to 500 microns.

Examples of tablet disintegrants useful in accordance with the present invention are starch, pregelatinized starch, sodium starch glycolate, sodium croscarmellose, microcrystalline cellulose, alginates, gums, surfactants, effervescent mixtures, hydrous aluminum silicate, cross-linked polyvinylpyrrolidone, and others as known in the art. Their concentrations are typically about 1% to about 8% by weight of formulation.

Examples of tablet binders include, e.g., acacia, cellulose derivatives (such as methylcellulose and carboxymethylcellulose, hydroxypropylmethylcellulose, hydroxypropylcellulose, hydroxyethylcellulose), gelatin, glucose, dextrose, xylitol, polymethacrylates, polyvinylpyrrolidone, starch paste, sucrose, sorbitol, pregelatinized starch, gum tragacanth, alginic acids and salts thereof such as sodium alginate, magnesium aluminum silicate, polyethylene glycol, guar gum, and the like. Their concentrations are typically about 5% to about 30% by weight of formulation.

A variety of materials may be used as fillers or diluents. Examples are spray-dried or anhydrous lactose, sucrose, dextrose, mannitol, sorbitol, starch (e.g. starch 1500), cellulose (e.g. microcrystalline cellulose; Avicel), dehydrated or anhydrous dibasic calcium phosphate, calcium sulfate, and others as known in the art. Their concentrations are typically about 10% to about 60% by weight of formulation.

Lubricants can also be employed herein in the manufacture of certain dosage forms, and will usually be employed when producing tablets. Examples of lubricants are magnesium stearate, zinc stearate, talc, glycerylbehenate, sodium lauryl sulfate, magnesium lauryl sulfate, sodium stearyl fumarate, colloidal silica, and others as known in the art. Preferred lubricants are magnesium stearate, and mixtures of magnesium stearate with sodium lauryl sulfate. Lubricants generally comprise 0.5 to 7.0% of the total formulation weight.

Other excipients such as glidants and coloring agents may also be added to atorvastatin calcium tablets. Coloring agents may include titanium dioxide and/or dyes suitable for food such as those known as F. D. & C, dyes and natural coloring agents such as grape skin extract, beet red powder, beta carotene, carmine, turmeric, paprika, and so forth. A coloring agent is an optional ingredient in the compositions of this invention, but when used will generally be present in an amount up to about 3.5 percent based on the total formulation weight.

Surfactants or other solubilizing agents may be used to aid the dissolution of drugs in the present invention. Suitable surfactant is sodium lauryl sulfate or Twin 80 and suitable solubilizing agents are PEG 400 and poloxamer. The preferred solubilizing agent according to the present invention is poloxamer. They will generally be present in an amount up to about 2 to about 5 percent based on the total formulation weight.

For making tablets, tablet blends may be dry-granulated or wet granulated before tableting. Alternatively, tablet blends may be directly compressed. The choice of processing approach depends upon the properties of the drug and chosen excipients, for example particle size, blending compatibility, density and flowability. For atorvastatin calcium tablets, granulation is preferred, with wet granulation being most preferred. The atorvastatin calcium, a water-insoluble alkaline excipient and an antioxidant may be wet-granulated, and then other excipients may be added extragranularly. Alternatively, the intimate admixture of atorvastatin calcium, a water-insoluble alkaline excipient and an antioxidant and one or more excipients may be wet-granulated, and then other excipients may be added extragranularly.

Pharmaceutical formulations such as tablets can be prepared by wet granulation and followed by compression. For instance, the ingredients are screened and blended in an industrial blender such as a high shear blender. The blended material was then granulated with water or suitable organic solvents. During the wet granulation, the contact time with water has to be very short, and granules are dried in a fluid bed drier. The concentrated granules and other excipients were further blended, and the mixture of ingredients are then compressed into tablets using, for instance, a Kikusui Libra® tablet compression machine.

Dry granulation, such as compaction and/or slugging with or without an intragranular excipient may also be used to make the tablets, followed by tabletting with or without extragranular excipients. In addition, tablets may also be coated, with a coating that exhibits little or no effect on or interference with tablet dissolution, to assure ease of swallowing or to provide an elegant appearance.

Tablets may be covered with a suitable coating. For example, the coating can be a moisture or oxygen barrier to help with storage stability or a sustained or delayed release coating composition as are well known in the art. Tablets may be film-coated to provide ease of swallowing and an elegant appearance.

The stability samples of the pharmaceutical formulation can be obtained in accordance with industry standards by storage for three (3) months at about 40° C. and about 75% relative humidity (RH). Standard analytical procedures such as HPLC methods may be used to determine the amount of active ingredient and degradants remaining after storage.

Suitable package material for packing the pharmaceutical dosage forms are plastic or glass containers and blisters. Particularly blisters made from non-permeable materials (high density polyethylene or aluminum) are advantageous as they may contribute to decreasing the rate of formation of degradants. Additionally, sufficient amount of desiccants, a minimum headspace to reduce the oxygen content in the packaging containers are preferred methods to package the pharmaceutical dosage forms comprising amorphous atorvastatin calcium.

The pharmaceutical formulation of the present invention can, if desired, also contain other compatible therapeutic agents. In particular, the pharmaceutical formulation can contain both atorvastatin calcium and additional therapeutic agents as active ingredients. However, the optimum dosage for the individual subject being treated will be determined by the person responsible for treatment, generally smaller doses being administered initially and thereafter increments made to determine the most suitable dosage.

More specifically, the invention also provides a use of atorvastatin calcium along with one or more additional agents. Since atorvastatin calcium is an HMG-CoA reductase inhibitor, a combination therapy with one or more cholesterol ester protein inhibitors or with one or more calcium-channel antagonists may be particularly desirable. The additional agents that can be used with atorvastatin calcium, for example, include amlodipine and its pharmaceutically acceptable salts, or torcetrapib and its pharmaceutically acceptable salts. Preferably, atorvastatin calcium is combined with amlodipine besylate or torcetrapib.

The additional agent may be combined with atorvastatin calcium to form a single stable pharmaceutical dosage form, for example, such as tablets or capsules or may be prepared as a separate pharmaceutical dosage form, which can be administered to a patient along with the pharmaceutical dosage form of atorvastatin calcium at the same time or with a time interval depending upon the patients conditions, and additional agents being used for the combination therapy. The pharmaceutical dosage form may include any forms of drug, which are suitable transport the therapeutic agents into body as noted hereinabove.

In a preferred aspect of the present invention, one example of such pharmaceutical dosage form of the combination therapy is a tablet, which may contain atorvastatin calcium and amlodipine besylate equivalent to: 2.5 mg amlodipine besylate with 10 mg atorvastatin calcium, 2.5 mg amlodipine besylate with 20 mg atorvastatin calcium, 2.5 mg amlodipine besylate with 40 mg atorvastatin calcium, 5 mg amlodipine besylate with 10 mg atorvastatin calcium, 5 mg amlodipine besylate with 20 mg atorvastatin calcium, 5 mg amlodipine besylate with 40 mg atorvastatin calcium, 5 mg amlodipine besylate with 80 mg atorvastatin calcium, 10 mg amlodipine besylate with 10 mg atorvastatin calcium, 10 mg amlodipine besylate with 20 mg atorvastatin calcium, in addition to inactive ingredients such as lactose monohydrate, magnesium stearate, microcrystalline cellulose, pregelatinized starch, sodium starch glycolate, titanium dioxide and one or more of red and yellow iron oxides.

A further aspect of the invention relates to a method of treating or preventing hypercholesterolemia and hyperlipidemia and prophylaxis of conditions associated with hypercholesterolemia and hyperlipidemia, including hypertension, cardiovascular disease, especially arteriosclerosis, and complication thereof in a mammal, comprising administering to a patient in need of such treatment an effective amount of a pharmaceutical composition comprising atorvastatin calcium, a water-insoluble alkaline excipient or a combination of two or more excipients thereof and an antioxidant or a combination of two or more antioxidants thereof, and plus one or more pharmaceutically acceptable inert excipients, comprising administering to a patient in need of such treatment an effective amount of atorvastatin calcium in such stable pharmaceutical formulation.

The following examples are illustrative of some of its embodiments of the present invention, but are not limiting the scope of the present invention. Modifications or variations will be apparent to one skilled in the art from consideration of the specification and examples.

### EXAMPLES

### Example 1

### Intimate Admixtures of Atorvastatin Calcium, Water-Insoluble Alkaline

### Excipients and Antioxidants

Intimate admixtures of atorvastatin calcium, water-insoluble alkaline excipients and antioxidants were prepared using co-precipitating and co-milling methods of admixing to assess their effectiveness at inhibiting degradation of atorvastatin calcium.

### Co-Precipitate Method

As shown in Table 1, crystalline or amorphous atorvastatin calcium, water-insoluble alkaline excipients and antioxidants were weighed out and then dissolved in absolute ethanol (50 ml) at 25° C. in a 250 ml three-necked flat flanged jacketed vessel equipped with a mechanical stirrer and a condenser. The ethanol solvent was evaporated under vacuum or reduced pressure at 25-35° C., until a dry residue is formed.

TABLE 1
Composition of intimate admixtures obtained
by co-precipitation method

| | Preparation 1 | Preparation 2 | Preparation 3 |
|---|---|---|---|
| Atorvastatin calcium | 10 (g) | 10 (g) | 10 (g) |
| Zinc carbonate | 10 (g) | | |
| Magnesium tribasic | | 10 (g) | |
| phosphate | | | |
| Calcium glycerophosphate | | | 10 (g) |
| Calcium sulfite | 0.30 (g) | | |
| Propyl gallate | | 0.20 (g) | |
| Ascorbyl palmitate | | 0.10 (g) | 0.30 (g) |

The dry product was dried under vacuum oven at 30-35° C. for 12-24 hours to remove ethanol solvent. The obtained products were packaged in induction-sealed HDPE bottles with desiccants and stressed at 40° C./75% RH for three months.

### Co-Milling Method

Crystalline atorvastatin calcium trihydrate (form 1) or amorphous atorvastatin calcium, water-insoluble alkaline excipients and antioxidants listed in Table 2 were weighed out, and were finely milled with a mortar and pestle. The atorvastatin calcium was added portion wise to water-insoluble alkaline excipients and antioxidants. Each portion was thoroughly milled with water-insoluble alkaline excipients and antioxidants using the mortar and pestle. The obtained intimate admixtures were packaged in induction-sealed HDPE bottles with desiccants and stressed at 40° C./75% RH for three month.

TABLE 2
Composition of intimate admixtures obtained by co-milling

| | Preparation 4 | Preparation 5 | Preparation 6 |
|---|---|---|---|
| Amorphous Atorvastatin | 10 (g) | 10 (g) | |
| calcium | | | |
| Atorvastatin calcium form I | | | 10 (g) |
| Zinc carbonate | 10 (g) | | |
| Magnesium tribasic | | 10 (g) | |
| phosphate | | | |
| Calcium glycerophosphate | | | 10 (g) |
| Calcium sulfite | 0.30 (g) | | |
| Propyl gallate | | 0.20 (g) | |
| Ascorbyl palmitate | | 0.10 (g) | 0.30 (g) |

### Preparations 7a, 7b and 7c (Control Experiments)

In this example, water-insoluble alkaline excipients and antioxidants were not used. In other respects, the atorvastatin calcium was processed according to co-precipitation (7a) and co-milling (7b for crystalline atorvastatin calcium, form I, 7c for amorphous atorvastatin calcium) methods and the resulting product was used as a control sample against which to compare the degradation rates of stabilized atorvastatin calcium compositions.

### Stability Results

Stability studies on batches produced according to preparations 1-6 were performed in a thermostated stability chamber adjusted to 40° C. and 75% of relative humidity (RH) in package material of induction-sealed HDPE bottles with desiccants. Assay of the active substance and the content of degradants was measured by HPLC method, using reference materials of atorvastatin calcium and their major degradants. The content of other detected unknown impurities or degradation products was calculated by internal area. In Tables 3, the assay of the active substance and total impurities are expressed in percentage. The results for initial and three month stability are recorded in Table 3.

The stability data shown in Table 3 clearly demonstrated that intimate admixtures of atorvastatin calcium, water-insoluble alkaline excipients and antioxidants, either prepared by co-precipitating or co-milling methods are dramatically more stable than the control samples. When the active ingredient is amorphous atorvastatin calcium (preparation 1-5), the stability effect of water-insoluble alkaline excipients and antioxidants is very dramatic. When the active ingredient is crystalline atorvastatin

TABLE 3
Stability of intimate admixtures made by co-precipitation and
co-milling at 40° C./75% RH for 3 months

| | | | | Total |
|---|---|---|---|---|
| | Assay | Total impurities | Assay | impurities |
| | (initial) | (initial) | (3 months) | (3 months) |
| Preparation 1 | 99.6 | 0.20 | 98.2 | 0.88 |
| Preparation 2 | 99.6 | 0.19 | 98.5 | 0.75 |
| Preparation 3 | 99.6 | 0.20 | 98.2 | 0.86 |
| Preparation 4 | 99.5 | 0.18 | 98.2 | 0.87 |
| Preparation 5 | 99.6 | 0.20 | 98.5 | 0.76 |
| Preparation 6 | 99.6 | 0.19 | 98.6 | 0.61 |
| Preparation 7a | 99.5 | 0.21 | 96.2 | 2.34 |
| Preparation 7b | 99.5 | 0.20 | 98.1 | 0.95 |
| Preparation 7c | 99.6 | 0.19 | 96.0 | 2.33 |

7a: control for co-precipitating method;
7b and 7c: control for co-milling method.

calcium (preparation 6), the stability effect of water-insoluble alkaline excipients and antioxidants is less dramatic, but it is still very significantly. For control samples comprising either amorphous or crystalline atorvastatin calcium without water-insoluble alkaline excipients and antioxidants (e.g., preparations 7a-c), significant amount of degradants was observed. Therefore, water-insoluble alkaline excipients and antioxidants dramatically improved the stability of amorphous atorvastatin calcium, and significantly improved the stability of crystalline atorvastatin calcium.

### Example 2

### Atorvastatin Calcium Tablets Prepared by Wet Granulation

There were three major steps involved in manufacturing the tablets: (i) preparation of atorvastatin calcium granular concentrate; (ii) preparation of atorvastatin calcium tablet core; (iii) coating the tablet core. The amount of each ingredient included in the formulation is shown in Table 4.

TABLE 4
% Composition of Atorvastatin Calcium (25%, w/w) Granular Concentrate

| | Granulation# | | | | | |
|---|---|---|---|---|---|---|
| Preparation# | A | B | C | D | E | F |
| Preparation 2 | 100 | | | | | |
| Preparation 3 | | 100 | | | | |
| Preparation 5 | | | 100 | | | |
| Preparation 6 | | | | 100 | | |
| Preparation 7b | | | | | 50 | |
| Preparation 7c | | | | | | 50 |
| Mannitol | 50 | 50 | 50 | 50 | 50 | 50 |
| Pregelatinized starch | 15 | 15 | 15 | 15 | 15 | 15 |
| Lactose anhydrous | 35 | 35 | 35 | 35 | 35 | 35 |
| Purified ethanol* | | | | | | |

*ethanol was removed during the process

(i): Preparation of Granular Concentrate

The following ingredients were sifted through a clean screen (typically 0.066″): excipients as shown in Table 4, intimate admixtures prepared in Example 1 (e.g., preparation 1-6), lactose anhydrous, mannitol and pregelatinized starch.

The screened materials were transferred into a high shear (high-energy) mixer and blended for ten (10) minutes at 100 rpm. The blended material was wet granulated with purified ethanol. The wet granules were passed through a screen (typically 0.132″), and dried in a fluid bed drier until loss on drying is less than 0.2-0.5% w/w.

The dried granules were passed a screen (typically 0.039″) and blended using a tumble blender for 10 minutes at 12 rpm.

(ii): Preparation of Tablet Core

The concentrated granules (granulation # A-F) are placed into a tumble blender. About two thirds of the lactose or mannitol is screened and added to the blender, and

TABLE 5
% Composition of Atorvastatin Calcium Tablet Core (quantity,
mg per tablet)

| | Formulation Batches # | | | | | |
|---|---|---|---|---|---|---|
| | 1 | 2 | 3 | 4 | 5 | 6 |
| Dosage strength | 20 mg | 20 mg | 20 mg | 20 mg | 20 mg | 20 |
| | | | | | | mg |
| Atorvastatin | A | C | B | D | E | F |
| calcium | 80 mg | 80 mg | 80 mg | 80 mg | 80 mg | 80 |
| concentrate | | | | | | mg |
| granules | | | | | | |
| Lactose | 50 | 50 | 50 | 50 | 50 | 50 |
| anhydrous | | | | | | |
| Sodium lauryl | 5 | 5 | 5 | 5 | | 5 |
| sulfate (SLS) | | | | | | |
| Microcrystalline | | | 40 | 40 | 40 | 40 |
| cellulose | | | | | | |
| Mannitol | 90 | 90 | 50 | 50 | 50 | 50 |
| Crosprovidence | 10 | 10 | 18 | 18 | 18 | 18 |
| Sodium starch | 8 | 8 | 0 | 0 | 0 | 0 |
| glycollate | | | | | | |
| Magnesium | 2 | 2 | 2 | 2 | 2 | 2 |
| stearate | | | | | | |
| Total weight of | 245 | 245 | 245 | 245 | 245 | 245 |
| tablet core | | | | | | |
| Coating material | 8.0 | 8.0 | 8.0 | 8.0 | 8.0 | 8.0 |
| (opadry) | | | | | | |
| Total weight of | 253 | 253 | 253 | 253 | 253 | 253 |
| coated tablet | | | | | | |

blended for ten (10) minutes. The crosprovidence, microcrystalline cellulose, sodium starch glycollate, magnesium stearate and remaining lactose or mannitol are screened and added to the blender. The mixtures are blended together for ten (10) minutes. The blended material was compressed on a Kikusui Libra tablet compression machine to a target weight of 200 mg for 20 mg tablets.
(iii): Preparation of Atorvastatin Calcium Coated Tablets

The tablet cores are then transferred to a tablet-coating machine (pan coater). The tablet bed was pre-heated with warm air (approximately 60° C.). The pan speed was adjusted to 5-9 RPM before starting the spray cycle. The spray cycle was activated. The exhaust temperature was maintained between 40° C. and 50° C. throughout the cycle. After the proper amount of solution was applied, the coated tablets were dried for approximately two (2) minutes. Steps were repeated for all pans to coat all tablets in the batch and film coated until the tablet weight has increased by 2.0% to 3.5%. All tablets were packaged in plastic bottles with desiccants, and the bottles were heat sealed, then placed under the stress condition.

### Example 3

### Stability Studies on Wet Granulated Atorvastatin Calcium Tablets

Stability studies on batches produced in Examples 2 were performed in a thermostated stability chamber adjusted to 40° C. and 75% of relative humidity in package material of induction-sealed HDPE bottles with desiccants. Assay of the active substance and the content of degradants was determined by HPLC method, using reference materials of atorvastatin calcium and their major degradants. The content of other detected unknown impurities or degradation products was calculated by internal area normalization. In Table 6, the assay of the active substance and total impurities are expressed in percentage.

The stability data from Table 6 indicated that above 98% w/w of initial potency of atorvastatin calcium was retained, and about less than 1.0% total impurities were formed

TABLE 6
Stability of wet granulated atorvastatin calcium tablets

| | Formulation Batches # | | | | | |
|---|---|---|---|---|---|---|
| | 1 | 2 | 3 | 4 | 5 | 6 |
| t = 0 month | | | | | | |
| Assay (potency, %) | 99.3 | 99.5 | 99.3 | 99.4 | 99.3 | 99.5 |
| Total impurities (%) | 0.28 | 0.30 | 0.32 | 0.28 | 0.30 | 0.28 |
| t = 3 months | | | | | | |
| Assay (potency, %) | 97.8 | 97.7 | 97.4 | 97.8 | 96.1 | 95.1 |
| Total impurities (%) | 1.15 | 1.21 | 1.29 | 1.05 | 1.75 | 3.02 |

after three (3) months at 40° C./75% RH in pharmaceutical formulation containing amorphous or crystalline atorvastatin calcium with a stabilizing-effective amount of water-insoluble alkaline excipients and a stabilizing-effective amount of antioxidants. At mean time, in pharmaceutical formulation #5 containing amorphous atorvastatin calcium without a water-insoluble alkaline excipient and an antioxidant, the total impurities were more than 3.02% w/w, and the active ingredient degraded significantly as determined by assay. At mean time, in pharmaceutical formulation #6 containing crystalline atorvastatin calcium without a water-insoluble alkaline excipient and an antioxidant, the total impurities were more than 1.75% w/w.

The data shown in Table 6 clearly demonstrated that the pharmaceutical formulations comprising intimate admixtures of amorphous or crystalline atorvastatin calcium, water-insoluble alkaline excipients and antioxidants, either prepared by co-precipitating or co-milling methods, when they are wet granulated with additional pharmaceutical inert excipients, are much more stable than the control formulations. In particular, atorvastatin calcium in control formulations #5 and #6 without water-insoluble alkaline excipients and antioxidants is much less stable than other stabilized formulations #1-4.

### Example 4

### Dry Granulated Atorvastatin Calcium Tablets

The stability of dry granulated tablet formulations of atorvastatin calcium that were pre-compressed by roller compaction was also assessed in formulations with and without an added water-insoluble alkaline excipient and food grade antioxidant.

Atorvastatin calcium was formulated into dry granulated 220 mg tablets following the stepwise procedure below using the excipients in Table 7. Part I materials were blended in a high shear mixer. Part II materials were added to the mixer and mixed. The mix was passed through a roller compactor. The compact was twice passed through the oscillating granulator. In the first pass, the granulator was equipped with a 2 mm aperture screen. In the second pass, the granulator was equipped with a 1 mm aperture screen. The

TABLE 7
Composition of Dry Granulated Atorvastatin Calcium Tablets

| | | Formulation (mg/Tablet) | | | | |
|---|---|---|---|---|---|---|
| Stage | Ingredients | 6 | 7 | 8 | 9 | 10 |
| Part I | Atorvsatin (form I) | 20 | | | | 20 |
| | Amorphous | | 20 | 20 | 20 | |
| | atorvastatin | | | | | |
| | Zinc Carbonate | | | 10 | | |
| | Magnesium phosphate | 20 | | | | |
| | Calcium | | 20 | | | |
| | glycerophosphate | | | | | |
| | Calcium phosphate | | | 10 | | |
| | Colloidal SiO₂ | 2 | 2 | 2 | 2 | 2 |
| | Vitamin E | | | 0.3 | | |
| | Ascorbyl palmitate | | 0.2 | | | |
| | Propyl gallate | | 0.2 | | | |
| | Calcium sulfite | 0.3 | | | | |
| | EDTA | | | 0.2 | | |
| Part II | Lactone anhydrous | 35 | 35 | 35 | 35 | 35 |
| | Mannitol | 123 | 123 | 123 | 145 | 145 |
| | Crosprovidence | 15 | 15 | 15 | 15 | 15 |
| | Croscarmelose sodium | 3 | 3 | 3 | 3 | 3 |
| | Magnesium stearate | 2 | 2 | 2 | 2 | 2 |
| Part III | Colloidal SiO₂ | 2 | 2 | 2 | 2 | 2 |
| | Sodium lauryl sulfate | 1 | 1 | 1 | 1 | 1 |
| | Magnesium stearate | 2 | 2 | 2 | 2 | 2 |
| Coating | Opadry | | | | | |
| Total weight | | 220.3 | 220.7 | 220.7 | 220.5 | 220.7 |

milled granulate was loaded a V-blender. The Part III materials were added to the V-blender and mixed. The mixed materials were compressed into tablet cores that was then coated Opadry white.

### Example 5

### Stability Studies on Dry Granulated Atorvastatin Calcium Tablets

Stability studies on formulation batches produced in Examples 4 were performed in a thermostated stability chamber adjusted to 40° C. and 75% of relative humidity in package material of induction-sealed HDPE bottles with desiccants. Assay of the active substance and the content of degradants was determined by HPLC method, using reference materials of atorvastatin calcium and their major degradants. The content of other detected unknown impurities or degradation products was calculated by internal area normalization. In Tables 8, the assay of the active substance and total impurities are expressed in percentage.

TABLE 8
Stability of dry granulated atorvastatin calcium tablets

| | Formulation Batches # | | | | |
|---|---|---|---|---|---|
| | 6 | 7 | 8 | 9 | 10 |
| t = 0 months | | | | | |
| Assay (potency, %) | 99.5 | 99.5 | 99.4 | 99.4 | 99.5 |
| Total impurities (%) | 0.28 | 0.30 | 0.31 | 0.28 | 0.28 |
| t = 3 months | | | | | |
| Assay (potency, %) | 97.3 | 97.4 | 97.5 | 94.6 | 96.0 |
| Total impurities (%) | 1.25 | 1.27 | 1.22 | 3.12 | 1.78 |

The stability data from Table 8 indicated that above 98% w/w of initial potency of atorvastatin calcium was retained, and about less than 1.3% total impurities were formed after three (3) months at 40° C./75% RH in pharmaceutical formulation containing amorphous or crystalline atorvastatin calcium with a stabilizing-effective amount of water-insoluble alkaline excipients and a stabilizing-effective amount of antioxidants. At mean time, in pharmaceutical formulation containing amorphous or crystalline atorvastatin calcium without water-insoluble alkaline excipients and antioxidants, the total impurities were more than 3.12% w/w and 1.78% respectively, and the potency of the active ingredient was significantly decreased.

The data shown in Table 8 clearly demonstrated that the pharmaceutical formulations comprising intimate admixtures of amorphous or crystalline atorvastatin calcium, water-insoluble alkaline excipients and antioxidants, either prepared by co-precipitating or co-milling methods, when they are dry granulated by roller compacting method with additional pharmaceutical inert excipients, are much more stable than the control formulation. In particular, control formulations #9 and #10, wherein the active ingredient is in an amorphous form or in crystalline form without water-insoluble alkaline excipients and antioxidants, is much less stable than other formulations #6, #7 or #8, wherein water-insoluble alkaline excipients and antioxidants were included in the pharmaceutical formulations.

We claim:

1. A stabilized pharmaceutical formulation comprising an intimate admixture or admixture of atorvastatin calcium, a water-insoluble alkaline excipient or a combination of two or more water-insoluble alkaline excipients, an antioxidant or a combination of two or more antioxidants, and at least one or more pharmaceutically acceptable inert excipients or carriers.

2. The stabilized pharmaceutical formulation according to claim 1, wherein less than about 3.0% degradants of atorvastatin calcium is formed on exposure to 40° C./75% relative humidity for three months.

3. The stabilized pharmaceutical formulation according to claim 1, wherein at least one or more pharmaceutically acceptable inert excipients or carriers is selected from the group consisting of a filler or a diluent, a binder, a disintegrating agent, a glidant, a lubricant, a surfactant, and a coating agent.

4. The stabilized pharmaceutical formulation according to claim 1, wherein the formulation is a form of a tablet, capsule, powder, granulate and suspension.

5. The stabilized pharmaceutical formulation according to claim 1, wherein the water-insoluble alkaline excipient has a pKₐ of conjugated acid of at least 2.5 and a Kₛₚ of about 1×10⁻¹ to about 1×10⁻¹⁵.

6. The stabilized pharmaceutical formulation according to claim 1, wherein the water-insoluble alkaline excipient is selected from the group consisting of zinc carbonate, zinc dibasic phosphate, zinc tribasic phosphate, cobalt carbonate, cobalt tribasic phosphate, calcium citrate, magnesium citrate, calcium glycerophosphate, magnesium glycerophosphate, sodium glycerophosphate, potassium glycerophosphate, magnesium dibasic phosphate, magnesium tribasic phosphate, magnesium carbonate hydroxide or calcium tribasic phosphate.

7. The stabilized pharmaceutical formulation according to claim 1, wherein the water-insoluble alkaline excipient is present in an amount of from about 0.5% to about 50% by weight of formulation.

8. The stabilized pharmaceutical formulation according to claim 1, wherein the antioxidant is selected from the group consisting of potassium ascorbate, calcium ascorbate and magnesium ascorbate, vitamin A, vitamin A₂, natural and synthetic tocopherols (mixed tocopherols concentrate, alpha-tocopherol, beta-tocopherol, synthetic gamma-tocopherol, synthetic delta-tocopherol), vitamin E, ascorbyl palmitate, ascorbyl stearate, propyl gallate, tertiary butyl hydroquinone (TBHQ), dilauyl thiodipropionate, magnesium sulfite or calcium sulfite.

9. The stabilized pharmaceutical formulation according to claim 1, wherein the antioxidant is present an amount of from about 0.01% to about 10% by weight of atorvastatin formulation.

10. The stabilized pharmaceutical formulation according to claim 1, wherein the atorvastatin calcium is amorphous atorvastatin calcium.

11. The stabilized pharmaceutical formulation according to claim 1, wherein the atorvastatin calcium is crystalline atorvastatin calcium.

12. A method of preparing the stabilized pharmaceutical formulation of claim 1, comprising steps of granulating an intimate admixture or admixture of atorvastatin calcium, a stabilizing-effective amount of a water-insoluble alkaline excipient and a stabilizing-effective amount of an antioxidant to form granules, mixing granules with additional excipients, and shaping said granules and additional excipients into a tablet.

13. The method of claim 12, wherein said granulating comprises wet granulation.

14. The method of claim 12, wherein said granulating comprises dry granulation.

15. The method of claim 12, wherein said granulating comprises roller compaction.

16. The method of claim 12, wherein the antioxidant is selected from the group consisting of potassium ascorbate, calcium ascorbate and magnesium ascorbate, vitamin A, vitamin A₂, natural and synthetic tocopherols (mixed tocopherols concentrate, alpha-tocopherol, beta-tocopherol, synthetic gamma-tocopherol, synthetic delta-tocopherol), vitamin E, ascorbyl palmitate, ascorbyl stearate, propyl gallate, tertiary butyl hydroquinone (TBHQ), dilauyl thiodipropionate, magnesium sulfite or calcium sulfite.

17. The method of claim 12, wherein the water-insoluble alkaline excipient is selected from the group consisting of zinc carbonate, zinc dibasic phosphate, zinc tribasic phosphate, cobalt carbonate, cobalt tribasic phosphate, calcium citrate, magnesium citrate, calcium glycerophosphate, magnesium glycerophosphate, sodium glycerophosphate, potassium glycerophosphate, magnesium dibasic phosphate, magnesium tribasic phosphate, magnesium carbonate hydroxide or calcium tribasic phosphate.

18. A stabilized intimate admixture comprising of amorphous or crystalline atorvastatin calcium, one or more water-insoluble alkaline excipients and one or more antioxidants.

19. The stabilized intimate admixture of claim 18, wherein the antioxidant is selected from the group consisting of potassium ascorbate, calcium ascorbate and magnesium ascorbate, vitamin A, vitamin A₂, natural and synthetic tocopherols (mixed tocopherols concentrate, alpha-tocopherol, beta-tocopherol, synthetic gamma-tocopherol, synthetic delta-tocopherol), vitamin E, ascorbyl palmitate, ascorbyl stearate, propyl gallate, tertiary butyl hydroquinone (TBHQ), dilauyl thiodipropionate, magnesium sulfite or calcium sulfite.

20. The stabilized intimate admixture of claim 18, wherein the water-insoluble alkaline excipient is selected from the group consisting of zinc carbonate, zinc dibasic phosphate, zinc tribasic phosphate, cobalt carbonate, cobalt tribasic phosphate, calcium citrate, magnesium citrate, calcium glycerophosphate, magnesium glycerophosphate, sodium glycerophosphate, potassium glycerophosphate, magnesium dibasic phosphate, magnesium tribasic phosphate, magnesium carbonate hydroxide or calcium tribasic phosphate.
